(19) United States
(12) Patent Application Publication
(10) Pub. No.: US 2024/0321461 A1
(43) Pub. Date: Sep. 26, 2024

(54) MAJOR ADVERSE CARDIOVASCULAR EVENT RISK PREDICTION BASED ON COMPREHENSIVE ANALYSIS OF CT CALCIUM SCORE EXAM

(71) Applicant: Case Western Reserve University, Cleveland, OH (US)

(72) Inventors: David L. Wilson, Cleveland Heights, OH (US); Sadeer Al-Kindi, Cleveland, OH (US); Yingnan Song, Cleveland, OH (US); Ammar Hoori, Westlake, OH (US); Hao Wu, Cleveland, OH (US); Yiqiao Liu, Cleveland Heights, OH (US)

(21) Appl. No.: 18/609,005

(22) Filed: Mar. 19, 2024

Related U.S. Application Data
(60) Provisional application No. 63/491,126, filed on Mar. 20, 2023.
(60) Provisional application No. 63/615,310, filed on Dec. 28, 2023.

(51) Int. Cl. G16H 50/30; G06T 7/00; G16H 15/00; G16H 50/20

(52) U.S. Cl. CPC G16H 50/30; G06T 7/0002; G16H 15/00; G16H 50/20; G06T 2207/10081; G06T 2207/20081; G06T 2207/30048; G06T 2207/30056; G06T 2207/30101

(57) ABSTRACT

Systems, methods, and apparatus are provided for determining a risk prediction for major adverse cardiovascular event (MACE) for a patient based on a computed tomography (CT) calcium score image of the patient's chest. In one example, a method includes receiving a computed tomography (CT) calcium score image of a chest; identifying tissue of interest in the CT calcium score image; analyzing the CT calcium score image to determine features of the identified tissue of interest; and determining a risk prediction of MACE based on the features.

### CROSS-REFERENCE TO RELATED APPLICATIONS

This application claims the benefit of priority from U.S. Provisional Patent Application Ser. No. 63/491,126 filed on Mar. 20, 2023 and entitled COMPREHENSIVE ANALYSIS OF CT CALCIUM SCORE EXAMS, and U.S. Provisional Patent Application Ser. No. 63/615,310 filed on Dec. 28, 2023 and entitled RADIOMICS-BASED RISK PREDICTION OF HEART FAILURE, the disclosures of which are hereby incorporated by reference in their entirety.

### BACKGROUND

A computed tomography (CT) calcium score exam uses CT to noninvasively score the amount of calcified plaque in coronary arteries. A higher score suggests a patient has more calcified plaque and correlates to a higher chance of a Major Adverse Cardiovascular Event (MACE). MACE may include conditions like myocardial infarction, death from coronary artery disease, coronary artery bypass graft (CABG), percutaneous coronary intervention revascularization, stroke, all-cause mortality, and heart failure.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates a flow chart of some embodiments of a method for comprehensive analysis of computed tomography (CT) calcium score exams.

FIG. 2 illustrates a flow chart of some embodiments of a method for comprehensive analysis of CT calcium score exams in which the method comprises preprocessing and report generation.

FIG. 3 illustrates a block diagram of some embodiments of a method for generating a risk prediction for MACE based on comprehensive analysis of CT calcium score exams.

FIG. 4 illustrates a block diagram of some embodiments of a method for generating a risk prediction for MACE based on comprehensive analysis of CT calcium score exams.

FIG. 5 illustrates a block diagram of some embodiments of a system for comprehensive analysis of CT calcium score exams.

### DETAILED DESCRIPTION

The description herein is made with reference to the drawings, wherein like reference numerals are generally utilized to refer to like elements throughout, and wherein the various structures are not necessarily drawn to scale. In the following description, for purposes of explanation, numerous specific details are set forth in order to facilitate understanding. It may be evident, however, to one of ordinary skill in the art, that one or more aspects described herein may be practiced with a lesser degree of these specific details. In other instances, known structures and devices are shown in block diagram form to facilitate understanding.

The present application comprises learning system methods for automated analysis of other findings in any electrocardiogram (EKG)-gated, non-contrast CT exam of the chest and targets the CT calcium score exam. Other findings are the opportunistic measurements that can be made from a CT calcium score exam, in addition to coronary artery calcifications (CAC). Gated, non-contrast CT calcium score exams have been obtained for several years, providing a wealth of potential information on the relationship between image assessments and disease. As large databases are available, this presents an opportunity to learn from the big data, immediate screening assessments of disease, and long term risk assessments of future disease or disease progression.

A host of opportunistic measurements can be made on CT calcium score images. Tissues and a brief description of assessments follow.

### Coronary Calcifications

Calcifications in the coronary arteries can be analyzed. The presence of calcifications indicates that there is coronary artery disease, as calcifications are not found in young healthy arteries. One potential assessment is an Agatston score, which is an important predictor of a future adverse cardiovascular event. Alternatively, individual calcifications are analyzed via a variety of computational assessments that are called calcium-related features. Calcium-related features are to be distinguished from a non-linear summary of all calcifications in the coronary arteries that corresponds to the Agatston score. As used herein, calcium-related features may be related to a large number of calcification features not considered in previous work related to CT calcium score images and/or MACE prediction, including, but not limited to, the following features. For each individual calcification, calcium-related features include mass, volume, territory, HU values, first moment, second moment, shape distance to subsequent lesion, distance to the top of the CT volume, artery diffusivity, among others. Artery diffusivity is the ratio of the number of calcified lesions to the Euclidean distance from the first to last lesion within an artery and represents the distribution of lesions within an artery. For a territory with no calcifications or a single calcification, diffusivity may be se to 0 or 1, respectively. For each coronary artery territory or for the entire heart, calcium-related features include statistical features such as mean standard deviation, skewness, kurtosis, and small histogram. For the entire heart, calcium-related features include total mass, total volume, peak HU, and average density.

### Aortic, Aortic Valve, and Mitral Annulus Calcifications

Extra-coronary calcifications in the aorta, aortic valve, and mitral annulus can be analyzed. The presence of calcifications indicates vascular disease with implications for coronary artery disease. The presence of calcifications in the aortic valve can also lead to future failure of the valve, thereby leading to intervention. Analysis of these individual calcifications may, for example, be done via calcium-related features.

### Liver Fat

Liver fat and potential fibrosis can be analyzed, thereby resulting in an important biomarker for nonalcoholic fatty liver disease (NAFLD) and possibly resulting in an indication of metabolic syndrome. In addition, liver fat is an independent indicator for cardiovascular disease. In the case of a CT calcium score exam, it makes sense to combine liver fat with calcium score to assess risk of cardiovascular disease. Assessments include fat concentration in the liver from CT intensity values. In addition to mean intensity values, morphometrics (e.g., shape, texture, etc.) and other CT intensity features (e.g., standard deviation, kurtosis, and histogram) are assessed for their association with cardiovascular risk. Textures may be related to the presence of fibrosis.

### Epicardial Fat

The visceral fat between the pericardium and the epicardial surface of the heart can be analyzed. Epicardial fat is an important source of pro-inflammatory mediators worsening endothelial dysfunction, eventually leading to coronary artery disease. Imaging biomarker assessments include morphometrics (e.g., volume and shape), CT intensity values, and textures, that are called fat-related features.

### Pericardial Fat

The visceral fat along the external surface of the pericardium can be analyzed since it is associated with cardiovascular disease. Pericardial adipose tissue is associated with increased risk of cardiovascular disease, blood glucose level, systolic blood pressure, hypercholesterolemia, and possibly atrial fibrillation. Assessments include morphometrics (e.g., volume and shape), CT intensity values, and textures (fat-related features).

### Subcutaneous Fat

The subcutaneous fat layer just under the skin can be analyzed. This is a biomarker of metabolic syndrome and is likely related to cardiovascular disease. Assessments include morphometrics (e.g., volume and shape), CT intensity values, and textures.

### Periaortic Fat

The fat surrounding the aorta can be analyzed. The presence of fat in this depot is a possible biomarker of cardiovascular disease. CT intensity of this fat depot is related to water content and inflammation. Assessments include morphometrics (e.g., volume and shape), CT intensity values, and textures.

### Pericoronary Fat

Fat surrounding the coronary arteries in the unenhanced CT images can be analyzed. Assessments include morphometrics (e.g., volume and shape), CT intensity values, and textures. Radiomics on pericoronary fat can be used for a good prediction of the risk of an adverse event. The opportunity is to enable such measurements in CT calcium score images.

### Heart Structures

Heart structures (e.g., whole heart, right atrium (RA), right ventricle (RV), left atrium (LA), left ventricle (LV), and aortic root) can be analyzed. Morphometrics (e.g., volume and shape) and possibly intensity textures are analyzed. These features may be early remodeling biomarkers indicative of future heart failure.

### Bone Density

Bone density from CT intensity values in the spine thoracic vertebrae can be analyzed. Bone mineral density is a marker of osteoporotic fractures, calcium metabolism, and cardiovascular disease. Morphometrics (e.g., volume and shape) and possibly intensity textures are assessed.

### Muscle

Skeletal muscle (e.g., pectoralis muscle) can be assessed for indications of sarcopenia and frailty. Measures of muscle loss (e.g., size, fatty replacement, etc.) are risk factors in all-cause mortality in patients with cardiovascular disease. CT intensity values, morphometrics (e.g., volume and shape), and intensity textures are assessed.

### Lung

Lung analysis provides a window into cardiovascular risk due to shared risk factors between lung disease and cardiovascular disease. Lung size, texture, and presence of nodules are analyzed as a marker of cardiovascular risk. Further, pulmonary artery shape and size are analyzed as a marker of pulmonary hypertension.

### Breast

Breast tissue analysis provides a window into cardiovascular risk. Breast arterial calcifications are associated with cardiovascular risk. Breast tissue morphometrics, texture, and presence/amount of calcifications are analyzed in women. These methods may, for example, be regarded as extensions of the above methods for coronary artery calcification analysis (e.g., using calcium-related features).

### Generalizations

It is understood that methods of the present application can involve only one of the items above or any combination of 2, 3, 4, or more items. It is also understood that some of the methods of the present application are difficult to fully automate accurately. In the software implementation, there will be an opportunity for manual identification of tissues for measurements and for editing of automated results. It is also understood that the procedures of the present application can be applied to other CT examinations (e.g., other than EKG-gated, non-contrast CT exams of the chest). For example, all procedures can be applied to any non-contrast, gated CT image of the chest. As another example, some procedures can be applied to any contrast, gated CT image of the chest with appropriate modifications for contrast. As another example, some procedures could be applied to non-contrast, non-gated CT images of the chest. As another example, some procedures could be applied to contrast, non-gated CT images of the chest with appropriate modifications for contrast.

### Processing Pipeline and Algorithms for Assessing Features

The methods of the present application comprise a complex processing pipeline with algorithmic innovations tuned for this problem.

### Inputs and Outputs

The input will be a DICOM CT image, such as a non-contrast, EKG-gated, CT calcium score examination. However, other EKG-gated CT exams are applicable. There will be multiple numerical outputs. The numerical outputs may comprise numeric measurements, disease probabilities, and risk predictions.

Numeric measurements include whole heart Agatston, liver fat, mineral bone density, volume of pericardial fat, and more. Disease probabilities include probability of existing disease (e.g., probability of fat inflammation, probability of NAFLD, etc.). Risk predictions are computed from single or a few measurements. For example, the risk of a cardiovascular event within 5 years will be determined from coronary calcifications or epicardial fat or from a combination of coronary calcifications and epicardial fat. In addition, risk will be computed from all or a large number of assessments possible in a CT calcium score exam, as described herein.

In addition, software will optionally create a professional report to a referring physician which can be shared with a patient. This report will include selected assessments, as described immediately above. In addition, the report will include an easy-to-understand description of topics like cardiovascular risk probabilities. The report is an important idea because, with proper patient education, there is an opportunity to improve patient adherence to potential drug (e.g., a statin to reduce lipids) or lifestyle change (e.g., smoking cessation, weight loss, and exercise). Showing patients images of CT calcium score improves adherence to statins and weight loss. One way to convey cardiovascular risk in an understandable way is to relate it to cancer risk or risk of driving a car. In addition, reports can indicate how the risk of disease might progress or stabilize as a function of life-style changes or drug therapies.

### Preprocessing

There are multiple, optional image preprocessing steps.

### Motion Artifact Suppression

The pipeline applies deep learning methods to improve data quality by minimizing cost function related to calcium motion. To train the deep learning network, a paired data set including moving and static images is generated by using a CT simulator by moving calcium with defined direction and velocity. The deep learning network uses moving image as input and generates output images with a minimized error to static images.

### Noise Reduction Using GAN

The pipeline applies generative adversarial network (GAN) to reduce noise due to low dose acquisition. Neural network learning methods may reduce noise in a variety of applications, including CT and low light imaging. A deep learning GAN may also reduce speckle noise in scattering-angle-resolved (SAR) images, optical-coherence tomography (OCT), and ultrasound. Methods may also be used to make ultrasound appear as CT. The algorithm is trained on a paired data set including low dose and high dose images which are generated from CT simulator, physical phantom, cadaver heart and clinical data with low and high acquisitions. Other embodiments can use advanced filtering such as nonlocal means or anisotropic diffusion.

Image volume normalization using a GAN method to make images look similar with regard to slice thickness and noise. Since data are acquired from different CT scanners and with different acquisitions (e.g., different dose level and slice thickness), the pipeline applies deep learning method to normalize data to improve quantification. Methods, such as GAN, may be applied to normalize structure and noise distributions on different CT acquisitions. To generate normalized volumes, the training data comprises CT volumes with different acquisitions (e.g., dose and slice thickness) and the reference volume. Different GAN models are trained for different acquisitions. Other embodiment can be using CycleGAN and other convolutional neural network (CNN) networks.

### Automated Beam Hardening Correction (ABHC)

Beam hardening correction is applied to reduce beam hardening artifacts which can otherwise be interpreted as low attenuated material and which may interfere with accurate and precise quantitative calcium score and fat analysis. The image-based ABHC algorithm automatically determines correction parameters for a beam hardening correction model and applies them to reduce artifacts in the image.

### Deconvolution for Coronary and Extra-Coronary Calcifications

In this case, three-dimensional (3D) deconvolution is applied on patient CT volumes to get more accurate measurement of coronary and extra-coronary calcifications. To perform deconvolution, a model for CT system degradation is assumed. For example, assume the CT system is linear and spatially invariant, whereby the output blurred image with additive noise is given below.

$$I ( x , y , z ) = \int \int \int f ( {x}^{′} , {y}^{′} , {z}^{′} ) h ( x - {x}^{′} , y - {y}^{′} , z - {z}^{′} ) {dx}^{′} d {y}^{′} x {z}^{′} + n ( x , y , z ) \text{} = f ( x , y , z ) * h ( x , y , z ) + n ( x , y , z )$$

Here, l(x, y, z) is the measured image volume from the CT system, f(x, y, z) is the idealized input image, ‘*’ denotes convolution, h(x, y, z) represents the 3D point spread function (PSF), and n is additive noise. The 3D PSF can be measured using a phantom, such as one containing very small metal beads. Alternatively, the PSF can be assumed to be a 3D Gaussian distribution, with parameters estimated from phantom with discrete objects or from a more general phantom or patient in an iterative solution.

The Lucy-Richardson deconvolution methods is used, which is an iterative approach with several important attributes. To estimate f(x, y, z), the method maximizes the likelihood of obtaining the output image data assuming Poisson noise statistics. The log likelihood is maximized in an iterative fashion. The PSF is assumed to be known, and the method constrains f to be non-zero. The method typically is reasonably fast and can be applied to three-dimensional problems. To reduce the effects of noise amplification, the method is modified to reduce noise. For example, a damping coefficient is applied as estimated from the noise in CT images. To reduce effects of noise even further, an anisotropic diffusion filter may be used.

There are many other approaches of deconvolution that can alternatively be used. Blind deconvolution can also be applied, whereby the PSF is estimated in addition to f. The iteratively obtained estimate of the PSF can be values stored in a 3D array which have been constrained in different ways (e.g., certain symmetries or non-negative values) or a 3D function.

### Segmentation of Important Tissues

Deep learning methods are used to identify the multiple tissues of interest. A potential solution uses a two-step approach. In step-1, the CT volume will be processed at a lower resolution to identify larger volumes of interest (VOIs) (e.g., heart and aorta; liver; and spine thoracic vertebrae including back musculature). In step-2, VOIs are processed via a deep learning semantic segmentation approach to identify actual tissues of interest. For example, the heart and aorta volume will be further processed to identify the cardiac chambers, epicardial fat, pericardial fat, and calcifications in the coronaries, aorta, and valves.

Deep learning semantic segmentation generally achieves better results than other automated methods for segmentation. Further, the entire CT image volume is not processed at full resolution using deep learning. Deep learning processing of the entire chest volume would be a challenge (if not impossible) on standard hardware. Further yet, by bringing attention to a VOI of interest, the network has an opportunity to better learn the local anatomy with a decreased number of training samples. It is understood that as time goes on, there will be improvements with hardware and network training paradigms which might surpass the method two-step approach above.

### Run-Time

At run-time (after training), the input will be a CT image volume. The output will be a labeled volume which identifies all tissue of interest (e.g., liver, epicardial fat, and muscle). At run time, a reasonably configured computer with a quality graphics card can be used. Training depends on more elaborate computational elements.

### Deep Learning Segmentation

Deep learning segmentation will be done in two steps. During training, step-1 and step-2 processing will be trained against appropriate ground truth, manual labels. At run time, outputs from step-1 will be processed in step-2.

Step-1. Segmentation of primary VOI bounding boxes of interest (heart and aorta; liver; and spine thoracic vertebrae including back musculature). Inputs are CT full volumes with 512×512 voxels in x-y plane and around 70 slices in z dimension and 0.7×0.7×2.5 millimeter (mm) resolution. Full volumes will be resampled to 256×256×35 voxels to form the input to a CNN. One CNN will be trained to segment each of the primary VOIs and background. An example CNN structure to be used is 3D U-Net with generalized Dice score as loss function. Three dimensional CNN is preferred over 2D CNN as more information can be learned. It is understood that other, possibly better 3D networks will be possible. Outputs are segmentation masks with each voxel belonging to one of the primary VOIs or background. Bounding box VOIs will be generated by cropping the full size CT volume to bounding boxes surrounding the segmentations.

Step-2. Refine segmentations of tissues of interest within each bounding box VOI. Each of the three bounding box VOIs will be processed independently. Depending upon the VOI, there will be a variable number of classes to segment. Since each voxel is classified to be one type of tissue, careful design of classes is needed. For the case of the liver VOI, the liver is accurately segmented at full resolution. In the case of the heart and aorta bounding box VOI, right atrium (RA), right ventricle (RV), left atrium (LA), left ventricle (LV); calcifications in the coronary arteries, aorta, aortic valve, and mitral annulus; and fat depots in epicardium, pericardium, and periaortic and pericoronary regions will be segmented. In one implementation, each bounding box VOI will have its own 3D U-Net.

### Deep Learning Details

A large, manually labeled dataset of CT image volumes is used for training and testing. In one implementation, active learning will be used whereby initial volumes are labeled and used to create initial segmentation software, which is then used to segment other volumes, which are then manually edited to provide a larger dataset for training and testing. In a typical scenario, image data will be split into training/validation/test by 80%/10%/10%.

As pre-processing, the dynamic range is shifted by +1,000 and the intensity is cut off at 0 so all values are non-negative. The intensity will be further normalized to the range of 0-1 after dividing the intensity by 2,000 and cutting the maximum off at 1. During the training of step-1, CT full volumes are resampled to size 256×256×35 to segment the primary VOIs. Data augmentation of scaling, rotation (±5°), translation, and 2D deformation are applied. Adam optimizer with a tentative learning rate of 10⁻⁴ is used. The training process is stopped if the loss function generalized Dice score doesn't increase for 10 epochs on validation dataset. Post processing with a conditional random field will be performed. The segmentation masks will be up-sampled to the full resolution as output and bounding box VOIs generated. During the training of step-2, a separate 3D U-Net is trained for each primary bounding box VOI. Bounding box VOIs are processed at full resolution if sufficient hardware is available. For data augmentation, small rigid body motions and small 3D deformations are used. Deep learning models are implemented using TensorFlow. Training is performed on NVIDIA DGXA100 cluster with four GPUs, where each GPU has 80 GB memory, or better. To evaluate the segmentation performances, Dice score, volume similarity, and Hausdorff distance are computed for all tissue labels of interest using the test dataset.

### Subcutaneous Fat

About 7 axial slices centered on a recognizable vertebra are identified using a regression deep learning approach. The subcutaneous fat is segmented in these image slices using a 2D deep learning CNN approach. A standard 2D CNN network is used to perform binary segmentation of subcutaneous fat and background. Much of the discussion above for 3D segmentation applies to this 2D segmentation problem.

### Analysis of Tissues of Interest

Analysis of tissues of interest is now described.

### Coronary Calcifications

Individual coronary calcifications are analyzed using advanced processing followed by risk prediction with machine learning. Measurements, such as whole heart Agatston, whole heart calcification mass, and aortic calcification mass, are reported. In addition, risk predictions, including predictions from calcium-related features different than those used to compute an Agatston score, are created.

### Aortic and Aortic Valve Calcifications

The presence of aortic valve calcifications is detected and a mass score is computed, using methods described for coronary calcifications. Briefly, calcifications are detected using a rule, such as candidate calcifications, located in a coronary artery, having at least 3 connected voxels exceeding 130 HU are only considered in assessments. A deep learning approach will identify calcifications in aortic value. The mass score of the calcification is then computed.

### Liver and Liver Fat Features

Liver fat and potential fibrosis are assessed, thereby creating an important biomarker for NAFLD and possibly giving an indication of Non-Alcoholic Steato Hepatitis (NASH). In addition, liver fat is deemed an independent indicator for metabolic and cardiovascular disease. In the case of a CT calcium score exam, it makes sense to combine liver fat with calcium score to assess risk of cardiovascular disease. Assessments will include fat concentration in the liver from CT intensity values. In addition, radiomics features, such as textures, in liver potentially associated with fibrosis, and surface nodularity, will be assessed.

### Liver Fat

Liver fat is estimated within the segmentation of the liver. As there is a potential for residual beam hardening, motion, and photon starvation artifacts, HU liver values samples within a 3D box containing a number of samples, NB, which will scan the liver with a small stride giving overlap of box assessments, are acquired. Within each box, at least mean, standard deviation, and kurtosis are computed. The 5 boxes having minimal standard deviation and kurtosis are selected and the median value across the 5 boxes is reported as representative HU of the liver. Multiple other realizations are possible (e.g., the mean or median over the entire liver could be computed, or the number of boxes used to estimate the central HU value of the liver could be changed). Another approach is to examine box values over a large number of CT image volumes and determine those box locations giving the most frequent result in a given liver across a large cohort. These locations can then be used to estimate the central HU value of the liver.

Once a representative HU of the liver is obtained, a value related to the amount of fat present in the liver is computed. There are multiple realizations: 1) report the HU value; 2) report the HU value divided by the value of the spleen; or 3) use an auto-calibration method as described below. Methods 2 or 3 are advised when CT images from older CT machines are analyzed, as in many risk prediction studies. In addition, when images are particularly noisy, there can be bias introduced in the mean value, thereby suggesting the desirability of using methods 2 or 3. In normal dose CT images, fatty liver is diagnosed if the value is less than 40 HU.

### Fat Value Auto-Calibration Method

HU values from somewhat noisy CT calcium score images maybe not be final arbiters of fat content in the liver. First, there is a tendency in noisy CT image data to introduce bias of the mean consisting of a few HU values, and this could be accentuated in a liver where there is increased body thickness. Second, there is a potential for machine drift, especially in older CT machines. Third, scatter and beam hardening can affect HU values. As such, in accordance with an auto-calibration method, reference tissues/structures consisting of air (in the surround), blood (in the aorta or ventricle), and spleen are automatically identified. A 3-point calibration curve is then applied to correct the offset. This auto-calibration method may, for example, be applied to correct any noise-dependent bias. Corrected values are suitable for identification of fatty liver.

### Liver Features

Within the liver region textural and surface features are computed as a potential assessment of liver fibrosis. Textural features of the liver may be obtained using non-enhanced scans. In alternative embodiments, textural features may be obtained with iodine enhanced images. Banks of texture features are used. Such texture features may, for example, include statistics of voxel histogram (e.g., mean, standard deviation, kurtosis, and entropy), Gray-level Co-occurrence Matrix, Local Binary Pattern, Laws, wavelets, and a variety of multi-orientation, multiscale filter banks (e.g., Gabor, MR8, and Leung-Malik). Machine learning will be applied to test the ability to predict clinical outcomes (e.g., incidence of diabetes, or major adverse cardiovascular events). An alternative to hand-crafted features is to use a deep learning approach. For example, a ResNet model network may be used. There are multiple deep learning networks which could be applied.

Diseased livers include surface irregularity, sometimes called nodularity. Methods for identifying surface irregularity can create additional features. One approach is to extract the surface from the image segmented liver, smooth the surface with a smoothing spline, compute corresponding distances between original and smoothed surface using a nearest point method, and then obtain statistics on the histogram of distances. Another method for identifying corresponding points is to points along radii from a central point in the liver obtained by fitting a sphere to the liver. Rather than using a smoothing spline, any number of other methods can be used to smooth a polygonal surface. Another assessment of nodularity is to create a plot of signed distances from the smoothed surface as described above and then analyze this 2D surface using texture metrics.

### Non-Exhaustive List of Additional Calcium-Related Features

Other calcium-related features may be collected from the CT Calcium Score image in addition to whole heart aggregated features (e.g., Agatston score, Volume score, and mass score) disclosed above. Lesion, lesion-to-lesion, and arterial-wise features may also be collected. Per-artery score features, including Agatston score, mass score, and volume score may also be calculated. Additional calcium-driven features include lesion aggregated areas, HU statistical features (e.g., min, max, average, median, and standard deviation), distance from the first slice to last calcification, and distance from first to last lesion along descending arterial lesions. Lesion-based statistical histogram bins of the first moment, second moment, mean moment, skewness moment, kurtosis moment, and average HU may be collected as well. The following is a non-exhaustive list of calcium-related features, where 2D features are slice based and 3D features are volume-based. Any of these features may be used as a factor in predicting MACE.

### Numerical Calcium-Related Features Include:

AgatstonScore3D (heart total Agatston score calculated in 3D volume-based lesions)
VolumeScorePerArtery_<<name>>1 (<<name>> artery volume score)
massHist<<number>> (histogram bin <<number>> out of 5 bins of lesions-based mass score)
avrHist<<number>> (histogram bin <<number>> out of 5 bins of mean HU values)
DistTop2LastLesionPerArtery_<<name>>1 (Euclidean distance summation in mm, starting from center of top CT slice along centroid of each consecutive lesion till last lesion within <<name>> artery)
DistFirst2LastLesionPerArtery_<<name>>1 (Euclidean distance summation in mm, starting from centroid of first lesion, along centroid of each consecutive lesion till last lesion within <<name>> artery)
ICfirstMomentH<<number>> (max values of first momentum among individual calcifications, order <<number>>) (<<number>> up to 3 values)
ICsecondMomentH<<number>> (max values of second momentum among individual calcifications, order <<number>) (<<number> up to 3 values)
ICmeanMomentH<<number>> (max values of mean momentum among individual calcifications, order <<number>>) (<<number> up to 3 values)
ICskewnessMomentH<<number>> (max values of skewness momentum among individual calcifications, order <<number>>) (<<number>> up to 3 values)
ICkurtosisMomentH<<number>> (max values of kurtosis momentum among individual calcifications, order <<number>>) (<<number> up to 3 values)
HUperArtery2D_stat<<name>><<number>> (<<number>> [1-4] represents [min, max, mean, std] statistical values of Hounsfield Units of each calcified voxel within artery <<name>>)
<<name>>_diffus (factor indicates diffusivity of lesions within <<name>> artery, calculated as the ratio of number of lesions to Euclidean distance along lesions with artery from first to last lesion. We considered the non-calcified artery to have zero diffusivity while the single lesion artery to have diffusivity=one)

### Categorical and Conditional (Boolean) Calcium-Related Features Include:

isLesion3DBelow5 (is number of lesions less than 5?)
HU1000 (Does the patient have any calcified lesions with HU value above 1000?)

### Non-Exhaustive List of Additional Fat-Related Features

Other fat-related feature may be collected from the CT Calcium Score image in addition to the fat-related features disclosed above. The fat-related features are collected based on measurements of heart structure and epicardial adipose tissue (EAT). The heart is equally divided into four axial slabs (e.g., Positional Quartiles (PQ)) from the top (PQ1) to the bottom (PQ4). EAT Hounsfield unit quartiles (HQ), ranging from 1 to 4, categorized HU values into bins: HQ1 includes values from −190 to −150, HQ2 includes values from −150 to −110, HQ3 includes values from −110 to −70, and HQ4 includes values ranging from −70 to −30. Spherical regions (SR) consisted of equidistant radial shells from the outside (SR1) to the inside (SR4) of the heart. The thickness of the heart was divided into four fixed histogram bins, each 8 mm wide. Based on these measurements, the following fat-related features may be identified. Any of these features may be used as a factor in predicting MACE.

### Structural Fat-Related Features

Total_SAC_Volume_Cm3 (total pericardial sac volume in cm3)
PrincipalAxisLength_max (major principal axis length of the pericardial sac)
PrincipalAxisLength_min (minor principal axis length of the pericardial sac)
PrincipalAxisLength_med (intermediate principal axis length of the pericardial sac)

### EAT Fat-Related Features

Total_Normalized_EAT (EAT volume/pericardial sac volume)
Norm Thickness_<<stats>> (<<mean, median, max, min, std>> of the EAT thickness divided by the corresponding radius in the same direction)
Thickness_bin<<number>>_Pro (probability of fixed thickness bins)
<<stats>>HU_PQ<<number>> (<<mean, median, max, min, std, kurtosis, skewness>> of the HU in each positional quartile <<number>> [1-4])
Vol_PQ<<number>>(volume of EAT in cm3 in each position quartile <<number>> [1-4])
<<stats>>HU_HQ<<number>>(<<mean, median, max, min, std, kurtosis, skewness>> of EAT HU in each HU bin HQ<<number>> [1-4])
PixelCount_HQ<<number>> (number of voxels of EAT in HQ<<number>> [1-4])
Probability_HQ<<number>> (probability of EAT Voxels with HU in HQ <<number>> [1-4])
Vol_HQ<<number>> (EAT volume in each HQ<<number>> [1-4])
Pro_HQ<<HU range>> (probability of EAT voxels in each HQ<<number>> [1-4])
SR<<number>>_Pro_<<HU range>> (probability of EAT voxels in HU ranges <<HU range>> ([−190, −170], [−170, −150], [−150, −130], [−130, −110], [−110, −90], [−90, −70], [−70, −50], [−50, −30]) in each spherical region <<number>> [1-4])

In addition to the above features, it is understood that features can also be extracted using deep learning methods.

### Learning System

A machine learning approach on hand-crafted features, a deep learning approach, or a hybrid approach is used. For hybrid learning, both hand crafted, and learned features from a deep learning network using a machine learning algorithm (e.g., random forest or SVM), are used. Such a machine learning approach can be trained to distinguish current or future NAFLD, current or future NASH, current or future diabetes, or future cardiovascular adverse event. An advantage is the ability to emphasize both convolutional features from deep learning with other assessments (e.g., distance along the vertebra giving a measurement of liver size and nodularity of the surface).

### Pericardial and Epicardial Fat Depots

There are two recognizable visceral fat depots external but in close proximity to the heart. The epicardial fat depot lies between the pericardium and the epicardial surface of the heart. It is in close proximity to the coronary arteries lying on the surface of the heart. Epicardial fat is an important source of pro-inflammatory mediators worsening endothelial dysfunction, eventually leading to coronary artery disease. The pericardial fat depot lies along the external surface of the pericardium. Pericardial adipose tissue is associated with increased risk of cardiovascular disease, blood glucose level, systolic blood pressure, hypercholesterolemia, and possibly atrial fibrillation. Pericardial and epicardial fat volumes are found to be highly correlated with adverse events. Deep learning can be used to segment the epicardial and pericardial fat depots in CT images. Steps for analysis are now described.

### Image Preprocessing

Low dose CT calcium score images can be noisy and suffer from motion artifacts, limiting the ability to visually and automatically separate boundaries of these two fat depots. At least in some images, it will be important to limit motion artifacts using the deep learning method described above. There are many methods for aggressive noise reduction. One implementation is non-local means and another is anisotropic diffusion. In both cases, parameters are adjusted for aggressive noise reduction.

### Automated Segmentation Fat Deposits

Two deep learning stages are used to segment heart surrounding fat tissues. Firstly, a deep learning approach is used to segment the heart from all the surrounding fat tissue, trained with a pre manually expert annotated mask. A deep learning segmentation method (such as deep attention Unet and DenseU-Net) is used. After excluding the heart mask and keeping the surrounding masked region only, a second, deep learning approach is used to segment the pericardial from the epicardial adipose tissues, with a pre annotated experts masking. Using two stage deep segmentation will lessen the intra-region overlapping, speed the convergence, and improve the results.

### Feature Assessment

Global assessments of the two fat depots include pericardial and epicardial fat volumes and their corresponding intensities (e.g., mean, standard deviation, kurtosis, and histogram). Treating each fat depot as a segmented “layer,” thickness measures are computed. Thicknesses will be determined for many points on the inner surface of the layer by finding corresponding points on the outer surface using a closest point algorithm. The result will be a set of thickness values with associated features determined from standard statistical measures (e.g., mean, standard deviation, minimum, maximum, kurtosis, and histogram). As such measures do not account for regional variation, and the layers will be mapped to a frame like that used to analyze perfusion data, which orients each patient's heart to vessel territories. A good example is an AHA 17-segment bulls-eye view. In this case, thickness values for a fat depot will be mapped to a bulls-eye view with 16 or 17 segments. Thickness statistics, volumes, and intensities can be obtained for each segment or for each vascular territory.

Altogether, a very large number of hand-crafted features are possible from each of the two fat depots. Fat features include pathologically driven hand-crafted features from EAT and PAT, which are carefully engineered to capture important information and organized into morphological, intensity, and spatial feature groups. Morphological features, like EAT volume, PAT volume and sac volume, were used to quantify volumes of interest. To analyze the spatial distribution of EAT and PAT within the heart region, the process includes subdividing the heart region into four equally thick slabs of image slices from top to bottom and 4 equidistant ribbons from outside to inside. Features are extracted from each of the 8 regions. Additionally, histogram range bins can be used to study the inflammation effect on certain locations of fat tissue, as it is known that increases in HU reflect the presence of inflammation in fat.

### Pericoronary Fat

Pericoronary fat is a good indicator of inflammation and of potential risk of an adverse event. Fat surrounding the coronary arteries in CT angiography (CTA) images may be analyzed. For example, a radiomic analysis can be performed on pericoronary fat in CTA images to predict the risk of an adverse event. The non-contrast CT images are used for this task instead. The challenge is to identify the coronary arteries in CT images. Images are aggressively processed to reduce noise to aid determination of the vasculature; vessel segments are segmented in the images; and pericoronary fat is analyzed using radiomics. Details follow.

### Noise Reduction Preprocessing.

Two options are non-local means and anisotropic diffusion with parameters adjusted for aggressive noise reduction.

### Semi-Automated Segmentation of Vessel Segments

The image volume will be processed with a vessel-ness filter to enhance blood vessels in 3D. In implementation-1, the user will specify the start and end of the vessel segment. The computer will identify the best vessel segment center-line connecting these points using a graphical optimization algorithm. 3D dynamic programming is one good option for finding the best path between the two endpoints. In implementation-2, the operator initiates a seed voxel in a vessel segment. Dynamic programing is run in the background to give a cost to each voxel in the image. The user then interactively identifies potential end voxels. This is similar to 2D methods described as “live-wire” and “intelligent scissors.” Both implementations will provide a center line approximation to a vessel segment. Repeated application of these approaches on vessel segments can segment the initial branches of a vessel tree.

### Automated Segmentation of Vessels

A deep learning approach is used within the heart VOI. Image inputs will include of both the original image volume and a processed volume where noise has been reduced and vessels have been enhanced with 3D vessel-ness filtering. A fully convolutional network (FCN) that accepts two image volumes is used. Training data comprises CT calcium score images and vessel labels. Vessel labels will be obtained from corresponding CTA images which have been processed using commercial methods to obtain vessel segmentations. Each CTA image volume is registered to its corresponding CT calcium score image volume using intensity based, 3D registration. This registration will enable mapping of the vessel label voxels to the CT calcium score image volume, providing the ground truth for deep learning. All appropriate deep learning approaches are applied.

Once trained, the method can be applied to new image data. The output of this semantic segmentation method will be a probability at each voxel of being a coronary artery voxel. The threshold applied to this volume will give vessels and eliminate noise. 3D connected components will be run. Connected components having a number of voxels less than the number for consideration will be eliminated. The result will be vessel segments suitable for processing. A 3D thinning operation will be used to get a vessel centerline.

### Segmentation and Analysis of Pericoronary Fat

Vessel center lines will be progressively dilated in 3D, creating a new shell around the centerline with each dilation. Within each shell, thresholds (e.g., HU between −190 and −30 HU) will be applied to segment the potential pericoronary fat. Dilation will stop at a predetermined value beyond which pericoronary fat is not thought to exist. The results will be a centerline and encapsulating pericardial fat. At this point, various features of the pericoronary fat are assessed. At each centerline location, fat areas and diameters will be obtained, as well as other morphometrics. Features based upon intensity values (e.g., mean, standard deviation, kurtosis, and histogram) will be assessed. Intensity gradients from the center line will be obtained as well as their statistics (e.g., mean, standard deviation, kurtosis, and histogram) will be obtained. Pericoronary fat features will be analyzed as described later.

### Heart Morphometrics

Heart structures (e.g., whole heart, RA, RV, LA, LV, and aortic root) are analyzed. This includes assessments of overall heart size (associated with mortality), LV hypertrophy (strongly associated with CV mortality), LA/RA size (strongly associated with heart failure and atrial fibrillation). These are challenging on non-contrast CT because visually the CT attenuation of blood and tissues is the same. So perhaps the morphometrics may be sufficient/feasible. Atrial/ventricular outer shape is readily identifiable. Morphometrics (e.g., volume and shape) and possibly intensity textures are assessed. These features could be potential early remodeling biomarkers indicative of future heart failure.

### Bone Density

We will assess bone density from CT intensity values in the spine vertebrae. Bone mineral density is a key modifiable risk factor for osteoporotic fractures which can be measured opportunistically in CT calcium score exams. We will assess bone mineral density using the auto-calibration method described above. This is an extension of the bone mineral density, phantom-less approach described previously. In addition to reporting bone mineral density, we will assess morphometrics (e.g., volume and shape) of vertebrae and intensity textures in a manner similar to described above for liver textures. In addition to bone mineral density values, there is an opportunity to use bone mineral density with or without other assessments in the spine to create a risk prediction of fracture within a time window such as 5 years.

### Muscle

Skeletal muscle is assessed for existing indications of sarcopenia. Also, muscle assessments are used to predict risks of later outcomes (e.g., all cause of mortality, adverse cardiovascular events, metabolic risk, and frailty). Among younger patients, it is determined whether there is a risk of later musculoskeletal issues. Sarcopenia (as estimated by pectoralis or vertebral muscle size and CT attenuation) are closely linked with outcomes in several cardiovascular conditions (heart failure and aortic stenosis). CT calcium score images will have a smaller volume of view than lung images, but nevertheless present an opportunity for muscle assessments. Muscles visible in axial images are assessed at different z-locations. Measurements will be reported in relation to vertebrae or other easily recognized anatomical structures. CT muscle intensity values will be assessed. CT HU values will be corrected using the auto-calibration method described above (Section Fat value auto-calibration method). Mean intensity will be reported. In addition, features are created from intensity histograms using standard deviation, kurtosis, histograms reduced to a few bins, and the like. Morphometric features (e.g., area and shape) are computed. Further, intensity texture features, as described above for other tissues, are computed.

### Numeric Measurements and Risk Predictions

Assessments described above are all amenable to a numeric measurement. For example, one can report Agatston score from coronary calcifications, calcification mass score in the aortic valve, volume of epicardial fat, calcification mass score excluding the coronaries, liver fat, bone mineral density, and more.

Another way to report such measurements will be to include probability of existing disease (e.g., probability of fat inflammation, probability of NAFLD).

In addition, one can make risk predictions created from a single or few measurements. For example, the risk of a cardiovascular event within 5 years will be determined from coronary calcifications, epicardial fat, or from a combination of coronary calcifications and epicardial fat. This will enhance the explainability of results. In addition, risk will be computed from all or a large number of assessments possible in a CT calcium score exam, as described herein.

### Risk Prediction Calculations

The assessments described above are referred to as “features”. To create a risk prediction depends on a set of features for each patient at a time point, the patient's outcome, and the time of outcome over a long time interval, say 5 years. A common outcome would be an adverse cardiovascular event (e.g., stroke, myocardial infarction, heart failure, arrhythmia, or coronary intervention). If the patient has not had an adverse event over the evaluation period, that would also be recorded. Such data can be plotted as a Kaplan-Meier and cox-regression models.

There are various methods to analyze such data in order to derive a probability of an event in 5 years. Further, each method allows for censoring (e.g., a patient who drops out at 4 years before having a positive event). The present application could use any suitable method. The methods include the Cox proportional hazards regression model, as well as machine learning approaches, including random survival forests and conditional inference forests. In some embodiments, clustering methods (e.g., K-means or hierarchical clustering) are used to identify novel cardio-metabolic phenotypes (e.g., calcific, frail, atherosclerotic, and lipodystrophic) and will identify their long-term outcomes. These phenotypes can serve as the bases for precision medicine approaches and targeted preventive strategies.

### Reporting

Software will optionally create a professional report to a referring physician which can be optionally shared with a patient. This report will include selected assessments, as described immediately above. In addition, the report will include an easy-to-understand description of topics like cardiovascular risk probabilities. With proper patient education, there is an opportunity to improve patient adherence to potential drug (e.g., a statin to reduce lipids) or lifestyle change (e.g., smoking cessation, weight loss, and exercise). Showing patients images of CT calcium score improves adherence to statins and weight loss. One way to convey cardiovascular risk in an understandable way is to relate it to cancer risk or risk of driving a car.

### Example Embodiments

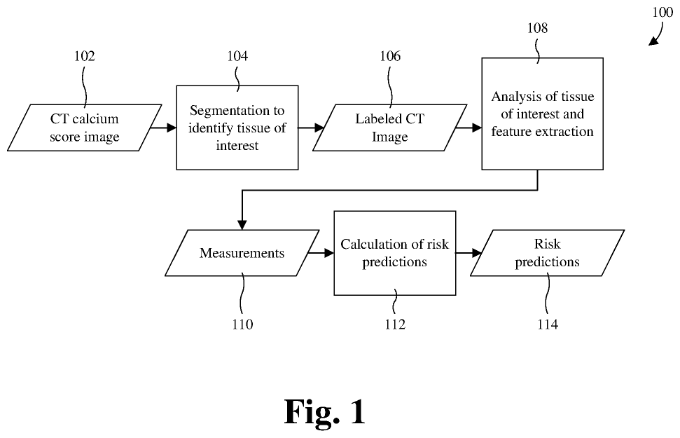

With reference to FIG. 1, a flow chart 100 of some embodiments of a method for comprehensive analysis of CT calcium score exams is provided. A CT calcium score image 102 of a chest is received or otherwise generated. The CT calcium score image may, for example, be an EKG-gated, non-contrast CT calcium score image or some other suitable type of CT calcium score image.

The CT calcium score image 102 undergoes segmentation 104 in which tissue of interest in the CT calcium score image 102 is labeled. Such tissue of interest may, for example, comprise: the liver; cardiac chambers (e.g., RA, RV, LA, and LV); calcifications in the coronary arteries; calcifications in the aorta; calcifications in the aortic valve; calcifications in the mitral annulus; fat depots in epicardium regions; fat depots in pericardium regions; fat depots in periaortic regions; fat depots in pericoronary regions; the like; all of the foregoing; or any subset of the foregoing. The segmentation 104 results in a labeled CT image 106.

The labeled CT image 106 undergoes tissue analysis 108. The tissue analysis and features extraction 108 extracts measurements 110 from the CT image. Such tissue analysis 108 may, for example, comprise: analysis of coronary calcifications; analysis of aortic calcifications; analysis of aortic valve calcifications; analysis of liver; analysis of liver fat; analysis of pericardial fat depots; analysis of epicardial fat depots; analysis of pericoronary fat; analysis of heart morphometrics; analysis of bone density; analysis of muscle; the like; all of the foregoing; or any subset of the foregoing. The extraction may, for example, be based on the tissue labels from the segmenting 104 and/or machine learning. In some embodiments, the tissue analysis further extracts disease probabilities (e.g., probabilities of a current disease).

One or more of the measurements 110 are used to calculate 112 risk predictions 114 using machine learning. In contrast with disease probabilities, the risk predictions 114 predict the likelihood of a patient developing a disease over an extended period (e.g., a period of 5 years or some other suitable period). Because gated, non-contrast CT calcium score exams have been obtained for several years, there are large databases available relating EKG-gated, non-contrast CT calcium score image to future disease or disease progression. These databases may be used to train the machine learning models used to calculate 112 the risk predictions 114.

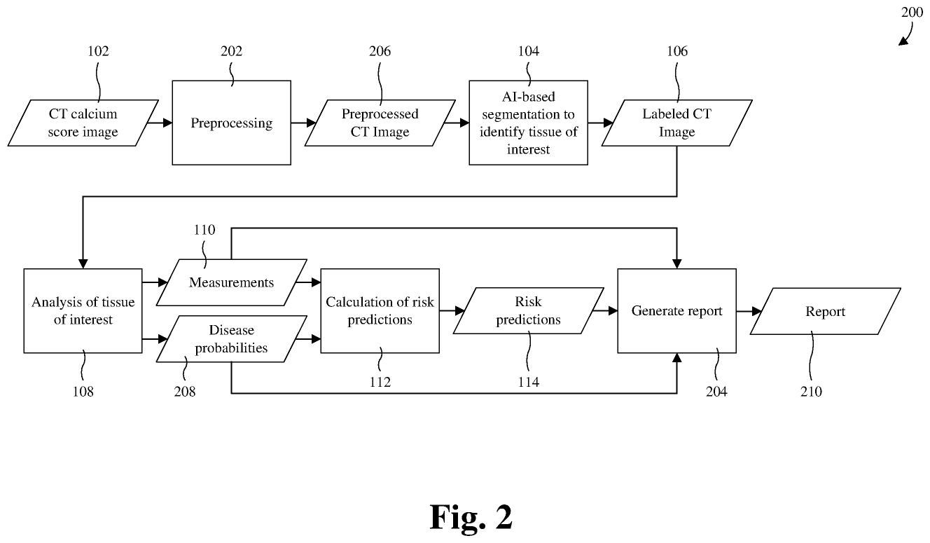

With reference to FIG. 2, a flow chart 200 of some embodiments of a method for comprehensive analysis of CT calcium score exams is provided in which the method comprises preprocessing 202 and report generation 204. In alternative embodiments, the preprocessing 202 and/or the report generation 204 is/are omitted.

The preprocessing 202 preprocesses the CT calcium score image 102 to enhance the CT calcium score image 102 into a preprocessed CT image 206. The preprocessed CT image 206 is used for subsequent processing (e.g., segmentation, analysis, etc.). The preprocessing 202 may, for example, include: motion artifact suppression; noise reduction using GAN; image volume normalization using GAN; ABHC; deconvolution; the like; all of the foregoing; or any subset of the foregoing.

The analysis 108 of tissue of interest results in the measurements 110 and further results in disease probabilities 208. As above, the disease probabilities are probabilities of a patient currently having a disease. In some embodiments, the disease probabilities 208 are used to calculate 112 the risk predictions 114. In other embodiments, the disease probabilities 208 are omitted from the calculation 112.

After the risk predictions 114 are generated, a report 210 is generated 204. The report 210 is based on the risk predictions 114 and, in some embodiments, the measurements 110 and/or the disease probabilities 208. The report 210 may, for example, be laid out in such a way that a lay-person (e.g., a non-clinician) can easily understand the report 210.

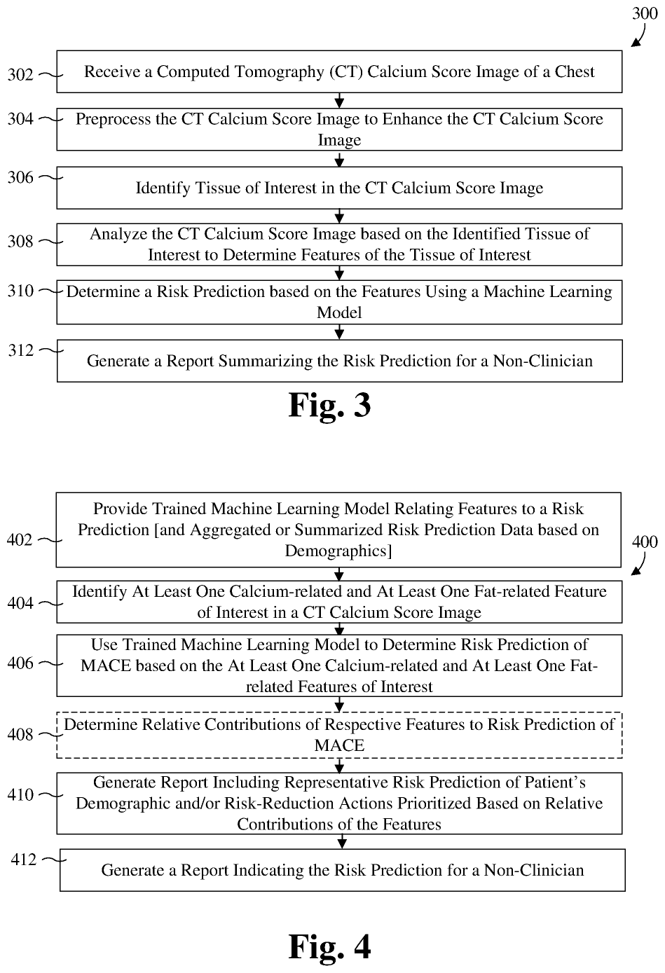

With reference to FIG. 3, a block diagram 300 of some embodiments of a method for comprehensive analysis of CT calcium score exams is provided.

At act 302, a CT calcium score image of a chest is received. In alternative embodiments, the CT calcium score image is generated from a CT scanner.

At act 304, the CT calcium score image is preprocessed to enhance the CT calcium score image. For example, noise may be removed, the image may be normalized, and so on. The preprocessing may, for example, be aided by machine learning models trained on datasets collected from past CT calcium score exams. In alternative embodiments, act 304 is omitted and the method proceeds to act 306.

At act 306, tissue of interest is identified in the CT calcium score image. The identification may, for example, be aided by machine learning models trained on datasets collected from past CT calcium score exams.

At act 308, the CT calcium score image is analyzed based on the identified tissue of interest to determine features of the tissue of interest. The features are numerical and the analysis may, for example, be aided by machine learning models trained on datasets collected from past CT calcium score exams.

At act 310, a risk prediction based on the features is determined using one or more machine learning models. The one or more machine learning models may, for example, be trained on datasets collected from past CT calcium score exams. One example method for risk prediction is time-to-event modeling that does the actual risk prediction. Time-to-event modeling can be done using Cox modeling (and variants, including elastic net) or deep learning time to event modeling.

At act 312, a report summarizing the risk prediction for a non-clinician is generated.

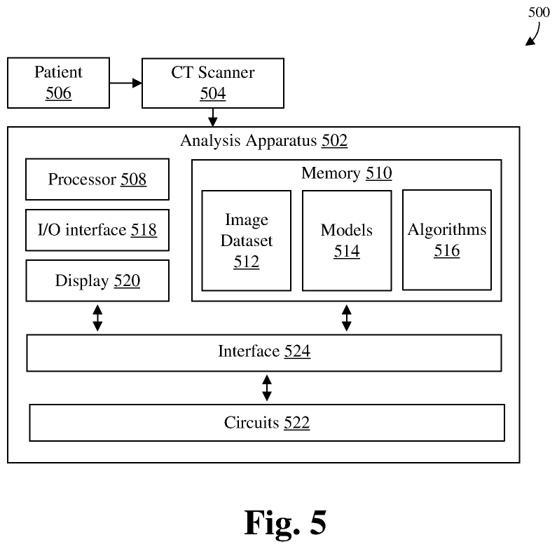

With reference to FIG. 4, a block diagram 400 of some embodiments of a method for generating a risk prediction for MACE based on comprehensive analysis of CT calcium score exams is provided.

At act 402, a trained machine learning model is provided that relates features of interest to a risk prediction for MACE. The trained machine learning model may be trained based on a training set of pre-processed CT calcium score images as described above. In some examples, demographic information including aggregated or summarized risk prediction values mapped to patient demographic is also provided for contextualizing the determined risk prediction for the patient.

At act 404, a CT calcium score image associated with a patient is received. In some examples, the image is a DICOM CT image. The image may be a non-contrast and EKG-gated CT calcium score examination.

At act 406, the CT calcium score image is pre-processed to identify at least one calcium-related feature of interest and at least one fat-related feature of interest. The at least one calcium-related feature of interest may include coronary calcifications, aortic calcifications, or aortic valve calcifications. The at least one fat-related feature of interest may include liver fat, pericardial fat, epicardial fat, periaortic fat, or pericoronary fat.

At act 408, optionally, a relative contribution of the at least one calcium-related feature of interest or the at least one fat-related feature of interest is determined. For example, a quantification of each feature of interest may be compared to a threshold value (e.g., and average value for the demographic) and then the features of interest may be ordered according to a difference between the feature of interest and the respective threshold. Alternatively, other mathematical analysis may be performed based on an algorithm (e.g., weighted sum of quantifications of features of interest) used by the machine learning model to generate the predicted risk to determine which feature of interest is the greatest contributor to the risk prediction. For example, in some patients fat-related features of interest may contribute more to the risk prediction than calcium-related features of interest.

At act 410, data related to the at least one calcium-related feature of interest or the at least one fat-related feature of interest is provided to the trained machine learning model to generate a risk prediction for MACE for the patient.

At act 412, a report indicating the risk prediction for MACE for a non-clinician is generated.

In some examples, the computed risk prediction may be contextualized in the report in a manner that is tailored to the patient. For example, a representative (e.g., average, 3 sigma range, and so on) risk prediction for a population to which the patient belongs (e.g., Hazard Ratio) may be presented in the report along with the patient's computed risk prediction. For example, if the patient is a 55 year old woman of certain ethnicity or race whose computed risk prediction of MACE is 15% in the next 5 years, stored aggregated risk prediction data may be accessed to determine a representative risk prediction (e.g., 5% in the next 5 years) for the overall population of 55 year old women of that ethnicity or race. The representative risk prediction may be provided alongside the computed risk prediction in the report. The demographic data used to determine the representative risk prediction may be generated a priori based on demographic data associated with CT calcium score images that were used to train the machine learning model. Alternatively, commercially available demographic data for MACE risk prediction may be accessed to determine the representative risk prediction.

In some examples, the report includes risk-reduction actions that may be taken by the patient to reduce the risk of MACE based on the comprehensive analysis of the CT calcium score image. Risk-reduction actions may include taking certain medications, certain dietary changes, weight loss, increasing cardiovascular activity or other types of exercise, and so on. To tailor the report to the patient, the relative contributions of the calcium-related features of interest and the fat-related features of interest determined at act 408 may be used to prioritize risk-reduction actions that affect the larger contributing factors. For example, certain medications may be efficacious in reducing calcium-related conditions. However, if the main contributor to the patient's risk prediction for MACE is fat-related, then weight loss may be more beneficial in reducing the risk of MACE than certain medicines that target calcium-related conditions. Likewise, if calcium-related conditions are the main contributor to the patient's risk prediction, then risk-reduction actions that target these conditions should be prioritized over risk-reduction activities associated with fat-related conditions.

While the disclosed methods 300 and 400 are illustrated and described herein as a series of acts or events, it will be appreciated that the illustrated ordering of such acts or events are not to be interpreted in a limiting sense. For example, some acts may occur in different orders and/or concurrently with other acts or events apart from those illustrated and/or described herein. In addition, not all illustrated acts may be required to implement one or more aspects or embodiments of the description herein. Further, one or more of the acts depicted herein may be carried out in one or more separate acts and/or phases.

With reference to FIG. 5, a block diagram 500 of some embodiments of a system for comprehensive analysis of CT calcium score exams is provided. The system is configured to generate risk predictions and, in some embodiments, a report summarizing the risk predictions, disease probabilities, and the like. The system comprises an analysis apparatus 502 coupled to a CT scanner 504. The CT scanner 504 is configured to act upon a patient 506 to generate an EKG-gated, non-contrast CT image of the chest or some other suitable CT image of the chest. The analysis apparatus 502 comprises a processor 508 and a memory 510.

The processor 508 can, in various embodiments, comprise circuitry such as, but not limited to, one or more single-core or multi-core processors. The processor 508 can include any combination of general-purpose processors and dedicated processors (e.g., graphics processors, application processors, etc.). The processor(s) 508 can be coupled with and/or can comprise memory (e.g., the memory 510) or storage and can be configured to execute instructions stored in the memory or storage to enable various apparatus, applications, or operating systems to perform operations and/or methods discussed herein.

The memory 510 can be configured to store an image dataset 512, machine learning models 514, and algorithms 516. The image dataset 512 comprises one or more anonymized radiological images from one or more patients. The one or more radiological images may, for example, correspond to CT images from past CT calcium score exams. Further, the image dataset 512 may, for example, comprise labels for the machine learning models 514. The one or more radiological images may be grouped into a training dataset for the machine learning models 514 and a verification dataset for the machine learning models 514. Each of the one or more radiological images can have a plurality of pixels or voxels, each pixel or voxel having an associated intensity.

The algorithms 516 implement the comprehensive analysis of CT calcium score exam images, as described above (e.g., at any one or combination of FIGS. 1-3). The algorithms 516 may, for example, utilize the machine learning models 514 to achieve risk predictions and/or the like.

The analysis apparatus 502 also comprises an input/output (I/O) interface 518 (e.g., associated with one or more I/O devices), a display 520, a set of circuits 522, and an interface 524 that connects the processor 508, the memory 510, the I/O interface 518, the display 520, and the set of circuits 522. The interface 524 can be configured to transfer data between the memory 510, the processor 508, the set of circuits 522, and external devices, for example, a medical imaging device such as a CT scanner or the like.

The set of circuits 522 can comprise hardware components for machine learning and/or the like. For example, the set of circuits 522 may be or comprise a graphics processing unit (GPU) and/or the like. The set of circuits 522 are configured to access the image dataset 512 to train the machine learning models 514 and/or to generate risk predictions using the machine learning models 514.

Examples herein can include subject matter such as an apparatus, including a CT system, a personalized medicine system, a processor, a system, circuitry, a method, means for performing acts, steps, or blocks of the method, at least one machine-readable medium including executable instructions that, when performed by a machine (e.g., a processor with memory, an application-specific integrated circuit (ASIC), a field programmable gate array (FPGA), or the like) cause the machine to perform acts of the method or of an apparatus or system for comprehensive analysis of CT calcium score exams, according to embodiments and examples described.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

“Computer-readable storage device”, as used herein, refers to a device that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, and other media from which a computer, a processor or other electronic device can read.

“Circuit”, as used herein, includes but is not limited to hardware, firmware, software in execution on a machine, or combinations of each to perform a function(s) or an action(s), or to cause a function or action from another logic, method, or system. A circuit may include a software controlled microprocessor, a discrete logic (e.g., ASIC), an analog circuit, a digital circuit, a programmed logic device, a memory device containing instructions, and other physical devices. A circuit may include one or more gates, combinations of gates, or other circuit components. Where multiple logical circuits are described, it may be possible to incorporate the multiple logical circuits into one physical circuit. Similarly, where a single logical circuit is described, it may be possible to distribute that single logical circuit between multiple physical circuits.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and the illustrative examples shown and described. Thus, this application is intended to embrace alterations, modifications, and variations that fall within the scope of the appended claims.

What is claimed is:

1. A method, comprising:
receiving a computed tomography (CT) calcium score image of a chest;
identifying tissue of interest in the CT calcium score image;
analyzing the CT calcium score image to determine features of the identified tissue of interest; and
determining a risk prediction of major adverse cardiovascular event (MACE) based on the features.

2. The method of claim 1, wherein the features include a feature other than coronary artery calcification.

3. The method of claim 1, wherein the features pertain to at least one of: coronary calcifications; aortic calcifications; aortic valve calcifications; liver; liver fat; pericardial fat depots; epicardial fat depots; pericoronary fat; heart morphometrics; bone density; or muscle.

4. The method of claim 1, wherein the determining comprises:
providing a machine learning model trained to relate the features to a risk; and
predicting the risk using the machine learning model, using calcium-related, fat-related, texture-related, intensity-related, or morphometrics-related features.

5. The method of claim 1, further comprising:
determining a relative contribution of the determined features to the risk prediction; and
generating a report summarizing the risk prediction, suggested risk-reduction actions prioritized based on the relative contribution of the determined features, percentile of similar group risk among population, or pictures of specific regions showing risk histograms.

6. The method of claim 1, further comprising:
preprocessing the CT calcium score image to enhance the CT calcium score image, wherein the identifying is performed on the CT calcium score image as enhanced.

7. The method of claim 6, wherein the preprocessing comprises at least one of motion artifact suppression, noise reduction, image volume normalization, automated beam hardening correction, or deconvolution.

8. The method of claim 1, wherein the identified tissue of interest comprises at least one of a liver, cardiac chambers, calcifications in coronary arteries, calcifications in an aorta, calcifications in an aortic valve, calcifications in a mitral annulus, fat depots in epicardium regions, fat depots in pericardium regions, fat depots in periaortic regions, or fat depots in pericoronary regions.

9. The method of claim 1, wherein the analyzing comprises at least one of: analysis of coronary calcifications; analysis of aortic calcifications; analysis of aortic valve calcifications; analysis of liver; analysis of liver fat; analysis of pericardial fat depots;
analysis of epicardial fat depots; analysis of pericoronary fat; analysis of heart morphometrics, analysis of bone density, or analysis of muscle.

10. The method of claim 1, further comprising
assessing bone mineral density from CT intensity values in spine vertebrae in the CT calcium score image; and
determining a risk prediction of fracture based on the assessments.

11. The method of claim 1, further comprising
assessing skeletal muscle intensity values in the CT calcium score image; and
determining a risk prediction of sarcopenia based on the assessments.

12. The method of claim 1, further comprising
determining the risk prediction based on coronary calcifications and epicardial fat detected in the CT calcium score image.

13. An analysis apparatus, comprising
a processor; and
memory storing a trained machine learning model that relates features of interest to a risk prediction for MACE, and instructions, that when executed by the processor, cause the processor to perform operations comprising
receiving a CT calcium score image associated with a patient;
processing the CT calcium score image to identify at least one calcium-related feature of interest or at least one fat-related feature of interest; and
providing data related to the at least one calcium-related feature of interest or the at least one fat-related feature of interest to the trained machine learning model to generate a risk prediction for MACE for the patient; and
generating a report indicating the risk prediction for MACE for a non-clinician.

14. The analysis apparatus of claim 13, wherein
the memory stores demographic information mapped to risk prediction for MACE; and
the instructions further comprise instructions, that when executed by the processor, cause the processor to perform operations comprising
identifying demographic information for the patient;
accessing the stored demographic information to determine a representative risk prediction for MACE for training patients in a similar demographic to the patient; and
including an indication of the representative risk prediction for MACE in the report.

15. The analysis apparatus of claim 13, wherein the instructions further comprise instructions, that when executed by the processor, cause the processor to perform operations comprising
determining a relative contribution of the at least one calcium-related feature of interest or the at least one fat-related feature of interest to the risk prediction; and
including suggested risk-reduction actions in the report, wherein the risk-reduction actions are prioritized based on the relative contribution of the at least one calcium-related feature of interest or the at least one fat-related feature of interest.

16. The analysis apparatus of claim 13, wherein the at least one calcium-related feature of interest includes a whole heart calcification mass or an aortic calcification mass.

17. The analysis apparatus of claim 13, wherein the at least one fat-related feature of interest includes liver fat, pericardial fat, epicardial fat, periaortic fat, or pericoronary fat.

18. A method, comprising:
providing a trained machine learning model that relates features of interest to a risk prediction for MACE;
receiving a CT calcium score image associated with a patient;
processing the CT calcium score image to identify at least one calcium-related feature of interest or at least one fat-related feature of interest; and
providing data related to the at least one calcium-related feature of interest or the at least one fat-related feature of interest to the trained machine learning model to generate a risk prediction for MACE for the patient; and
generating a report indicating the risk prediction for MACE for a non-clinician.

19. The method of claim 18, comprising:
identifying demographic information for the patient;
determining a representative risk prediction for MACE for training patients having similar demographic information to the patient; and
including an indication of the representative risk prediction for MACE in the report.

20. The method of claim 18, comprising:
determining a relative contribution of the at least one calcium-related feature of interest or the at least one fat-related feature of interest to the risk prediction; and
including suggested risk-reduction actions in the report, wherein the risk-reduction actions are prioritized based on the relative contribution of the at least one calcium-related feature of interest or the at least one fat-related feature of interest.

21. The method of claim 18, wherein the at least one calcium-related feature of interest includes a whole heart calcification mass or an aortic calcification mass.

22. The method of claim 18, wherein the at least one fat-related feature of interest includes liver fat, pericardial fat, epicardial fat, periaortic fat, or pericoronary fat.
